(19) United States
(12) Patent Application Publication
(10) Pub. No.: US 2005/0043897 A1
(43) Pub. Date: Feb. 24, 2005

(54) Biometric compatibility matching system

(76) Inventor: Robert W. Meyer, Oviedo, FL (US)

Correspondence Address: Robert W. Meyer, 3472 Scout Lake Lane, Oviedo, FL, 32765, US

(21) Appl. No.: 10/913,924

(22) Filed: Aug. 7, 2004

Related U.S. Application Data
(60) Provisional application No. 60/493,967, filed on Aug. 9, 2003.

(51) Int. Cl. G01N 33/48; G06G 7/48

(52) U.S. Cl. 702/19; 703/11

(57) ABSTRACT

New information and amusement is provided by a method for determining compatibility and resemblance between people, by using biometric face recognition to compare facial features. A computer server operates as either connected via the Internet or as a non-networked, standalone kiosk. Facial image data is received from a user and compared to other stored images to compare similarities. Processing is performed on the computer server by the face recognition module and application software. A determination is then made as to compatibility and/or the resemblance level between facial images. Compatibility and resemblance are each directly correlated to how closely the user's facial features match those of other stored images. The computer server input/output module then outputs the results to the user as an analysis containing match scores.

### RELATED APPLICATIONS

The present application is a continuation-in-part application of U.S. provisional patent application Ser. No. 60/493,967, filed Aug. 9, 2003, for BIOMETRIC COMPATIBILITY MATCHING, by Robert W. Meyer, included by reference herein and for which benefit of the priority date is hereby claimed.

### FIELD OF THE INVENTION

The present invention relates to a computer-implemented dating service, and more particularly, to a system, method and computer program products for real-time, online computer searching and matching of database entries based on facial resemblance between two or more individuals, derived by using biometric face recognition software to compare facial features, and other user-selectable search criteria.

### BACKGROUND OF THE INVENTION

My invention relates to a method for determining compatibility and resemblance between individuals by using biometric face recognition to compare facial features. This invention is based upon my premise that two people, who share common facial features, will often feel more comfortable with each others company, and are therefore more compatible with each other. Although this concept may have many practical real-world applications, the primary focus here is to describe its use as an automated tool for the purpose of quickly matching compatible adults seeking companionship, dating or romance. Three additional capabilities of this invention, all of which also using face recognition, will be described herein as well. All services described herein can be subscriber based or paid for on an individual basis.

“Fully, 64.1% of all people believe that love at first sight can happen! Of these believers, 58.2% have fallen in love with someone within sixty minutes of meeting them.” Note 1

It is my contention that one or more similar facial characteristics is what attracts these individuals. For example, the very different backgrounds of Arnold Schwarzenegger and wife Maria Shriver (Republican/Democrat, 10 year age difference, family backgrounds, body types, country of origin, etc.) appear to make them a very unlikely couple. But when considering their very similar facial features, especially their distinctive cheekbones and unique jaw lines, the attraction seems much more obvious. Ever notice how often other couples closely resemble each other too?

It is also my contention that this attraction does not typically occur at a conscious level, and can therefore be aided by an automated process. Facial recognition software has the capability to measure many specific facial characteristics, and then quickly scan a large database searching for people with similar features.

This invention also incorporates 3 additional novelty or “human-interest” capabilities that use facial recognition as their key component. These additional capabilities are:

1) Family/Friends Matchup—Matching a group of family/friends to compare how closely they resemble each other. Photos are submitted and each photo is compared to every other photo within that particular family/friends group. A score is generated for each comparison to indicate how closely each group member resembles the other members. How often have you heard comments such as “He looks more like his father”, or “Who do you think she resembles most?” This invention will answer those types of questions.

2) Celebrity Matchup—Matching a person against a database of celebrities to determine the closest matches.

Celebrities include well-known public figures such as athletes, politicians, TV, movie, music and any other famous people; living or not. The database of celebrity photos may also include several photos of the same celebrity, such as a “young Paul Newman” and an “older Paul Newman”.

3) Pet Matchup—Matching a person to a pet species, such as all dog breeds for example, to determine the closest matching breeds to the user. Although admittedly for amusement purposes, statistical data relating to a large sample of human faces and all dog breeds is gathered and compared. The result is the closest matching dog breed to the user. Ever notice how people sometimes resemble their pets?

Facial recognition software falls into a larger group of technologies known as biometrics. The basic idea behind biometrics is that our bodies contain unique properties that can be used to distinguish us from others. Facial recognition methods may vary, but they generally involve a series of steps that serve to capture, analyze and compare a face to one or many other faces.

If you look in the mirror, you can see that your face has certain distinguishable landmarks. These are the peaks and valleys that make up the different facial features. There are about 80 of these landmark points on a human face. A few of these points that are measured by face recognition software are: distance between eyes; width of nose; depth of eye sockets; cheekbones; jaw line; chin, etc.

These points are measured to create a unique numerical code that represents the face in a database. This numerical code is called a faceprint. Once the system has stored a faceprint, it can then compare it to the thousands or millions of faceprints stored in a database. As comparisons are made, the system assigns a score to each comparison to indicate how close the match is. Potential matches are found when a score is above a predetermined threshold.

While humans have had the innate ability to recognize and distinguish different faces for millions of years, computers are just now catching up.

Notes:
1. Earl Naumann, Ph.D., Love At First Sight: The Stories and Science Behind Instant Attraction (Naperville, Ill.: Casablanca Press, 2001).

References Cited—U.S. Patent Documents

A handpaper search was conducted at the U.S. Patent Office. Computer database searching was done using the Patent & Trademark Office (PTO) patent search system. These searches were conducted through the Law Offices of Brian S. Steinberger, P.A. of Cocoa Florida, and also by the inventor.

Four (4) utility patents and five (5) published patent applications were found and analyzed:
U.S. Pat. No.: 5,963,951 10/1999 Collins; Gregg
U.S. Pat. No.: 6,507,912 01/2003 Matyas, Jr., et al
U.S. Pat. No.: 6,606,398 08/2003 Cooper
U.S. Pat. No.: 6,721,733 04/2004 Lipson, et al
Pub. No.: US 2003/0039410 Al 02/2003 Beeman, et al
Pub. No.: US 2003/0108225 Al 06/2003 Li
Pub. No.: US 2003/0161505 Al 08/2003 Schrank
Pub. No.: US 2004/0008874 Al 01/2004 Koike
Pub. No.: US 2004/0010608 Al 01/2004 Piccionelli, et al

Although others have invented biometric face recognition, and others have invented various aspects of online dating services, my invention is unique, novel or superior because it:
Uses facial recognition technology for the first time in public as a key component of an online dating service.
Concentrates on similarities between user's features, not necessarily exact matches, to determine compatibility level.
Allows for very precise computer-aided biometric measurements of each user's facial features to objectively determine the degree to which individuals match each other.
Can be added as a feature or component to an existing online dating service.
Compatibility matching works well with all races, genders, and hair/beard styles.
Quickly compares a user's faceprint to a database of potentially millions of faceprints in seconds.
Provides a certain level of entertainment value by displaying pictures of people with facial features similar to those of the user.
Provides a method for users to feel scientifically matched up for compatibility.
Uses facial recognition technology for the first time in public to objectively evaluate the facial resemblance strictly between a group of family and/or friends to determine the closest matches.
Uses facial recognition technology for the first time in public to compare a user's facial features in order to search for celebrities with the closest resemblance.
Uses facial recognition technology for the first time in public to compare a user's facial features in order to search within a species (e.g. dog) for a breed (e.g. bulldog) with the closest resemblance.
A self-contained, standalone kiosk embodiment allows for use in locations such as shopping malls, arcades, amusement parks, and other public locations.
Provides search results and/or a comparative analysis of facial features to the user.
As technology progresses, easily allows a user to select which facial features to concentrate the search on e.g. eyes, nose, mouth, roundness of face, jaw line, etc., and combinations thereof).
As technology progresses, easily allows a user to select predefined default facial features to concentrate the search on.
As technology progresses, can compare one user to one other user (one-to-one) for an analysis of how closely their specific facial features match.

It is therefore an object of the invention to provide a system and method of determining compatibility and resemblance between people by using biometric face recognition to compare facial features.

It is another object of the invention to provide a method of computerized facial matching of individuals for purposes of possible companionship, dating or romance.

It is another object of the invention to provide a method of running a computerized online dating service for matching people by using biometric face recognition.

It is another object of the invention to provide a method of comparing a group of family/friends to each other, seeking similar facial features, to determine how closely they resemble one-another.

It is another object of the invention to provide a method of entertainment by comparing a person to a database of celebrities to determine which well-known people he/she most closely resembles.

It is another object of the invention to provide a method of entertainment by comparing a person to a database of dog breed photos to determine which breed the user most closely resembles.

It is another object of the invention to provide a method of incorporating an add-on feature for a computerized online dating service to match people by using biometric face recognition.

It is another object of the invention to provide a method of automating “love-at-first-sight” by selectively looking for one or more similar facial features between individuals from a database of people.

It is another object of the invention to provide a method of comparing people one-to-one, seeking similar facial features, to determine level of compatibility, or to just compare the resemblance.

It is another object of the invention to provide a method of comparing people one-to-many, seeking similar facial features, to determine level of compatibility.

It is another object of the invention to provide a method of entertainment by displaying pictures of people with facial features similar to those of the user.

It is another object of the invention to provide a method of entertainment by performing one-to-one analysis of facial features of 2 people to compare the resemblance.

It is another object of the invention to provide a method of allowing users to feel scientifically matched up for compatibility.

It is another object of the invention to provide a method of compatibility matching which works well with all races, genders, and hair/beard styles.

It is another object of the invention to provide a method of displaying the best matching search results and/or analysis to the user.

### SUMMARY OF THE INVENTION

In accordance with the present invention, there is provided a system and method for determining compatibility and resemblance between people by using biometric face recognition to compare facial features.

In the Dating Matchup embodiment, the invention is structurally described as follows. A computer server is connected to a network such as the Internet. The computer server contains a server database for data storage; face recognition module for processing unique faceprints; applications software for general application processing; and input/output module to handle all data coming into and out of the computer server. User's photo(s) are required for all embodiments.

In this embodiment, the invention is functionally described as follows. User's photo(s) and other information (age, gender, etc.) are uploaded directly via the Internet to the computer server where an input/output module receives them. The application software and face recognition module then process the photo(s) automatically. The user's faceprint and other information are then stored in the server database immediately, with no manual processing required. Other user's photo(s) and other information are all processed similarly.

To search the server database for compatible members, a user must first logon and provide search criteria. These search criteria can include various combinations of information stored in the server database. The computer server then performs a server database search for the closest matching entries based on all specified search criteria. As is the primary purpose of this invention, this includes a search for all people whose facial features most closely match the user's own facial features. Searches may be accomplished without regard to race, gender, and hair/beard styles.

The closest matching entries are then generated as a result of the database search, with the closest matching results listed first. The application software and face recognition module generate this display page, which varies depending on which embodiment is being used. The input/output module then sends these results via the Internet to the user. The user then has the option to rerun the search using alternate search criteria, or to be done.

Alternative embodiments allow the system to be implemented as either networked (e.g. via Internet) or as a non-networked, standalone kiosk. These embodiments are functionally described as follows:

Family/Friends Matchup allows the submission of photos representing a group of family and/or friends. Each of the photos is processed into a unique faceprint, and then each faceprint is compared to every other faceprint within the group to obtain a match score for each comparison. The result is an analysis and report of how closely each person in the group resembles every other person within that group. For example, the results would show whether the son looks more like the father or the mother.

Celebrity Matchup allows a user to compare himself to a database containing the photos of well-known public figures such as athletes, politicians, actors, music and any other famous people; living or not. The purpose is to compare the user's face to all or a subset of celebrities, in order to determine which celebrities most closely resemble the user. A subset of all celebrities could be used to narrow the results to a specific category, such as actors. The result is a list of photos, match scores, and brief biographical data for the closest celebrity matches for the user's selected category.

Pet Matchup allows a user to compare himself to a database containing the photos of a pet species, such as all dog breeds for example. To do this, statistical data is gathered to determine upper/lower boundaries for facial features such as distance between eyes, nose size, face roundness, etc. The same gathering of statistical data for all dog breeds for example, is also compiled. Then, when doing the comparison between a human and a pet species, the faceprint and statistical data are compared to determine the closest matching breeds to the user. For example, if the user has a statistically small nose, a breed of dog with a statistically small nose would be returned as one of the match results. The result is a list of photos, match scores, and brief description for each of the closest breed matches for the user's selected species.

Additional embodiment features and variations are described in the Detailed Description section.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a block diagram view of an example Overall System Architecture;

FIG. 2 is a flow chart view of a Data Input operations that comprise the method;

FIG. 3 is a flow chart view of a Dating Matchup operations that comprise the method;

FIG. 4 is a flow chart view of a Family/Friends Matchup operations that comprise the method;

FIG. 5 is a flow chart view of a Celebrity Matchup operations that comprise the method;

FIG. 6 is an illustration view of an example Dating Matchup Summary Results display page that comprises the method;

FIG. 7 is an illustration view of an example Dating Matchup Photo Gallery Results display page that comprises the method;

FIG. 8 is an illustration view of an example Family/Friends Matchup Results display page that comprises the method;

FIG. 9 is an illustration view of an example Celebrity Matchup Results display page that comprises the method; and

FIG. 10 is an illustration view of an example Pet Matchup Results display page that comprises the method.

For purposes of clarity and brevity, like elements and components will bear the same designations and numbering throughout the FIGURES.

### DESCRIPTION OF THE PREFERRED EMBODIMENT

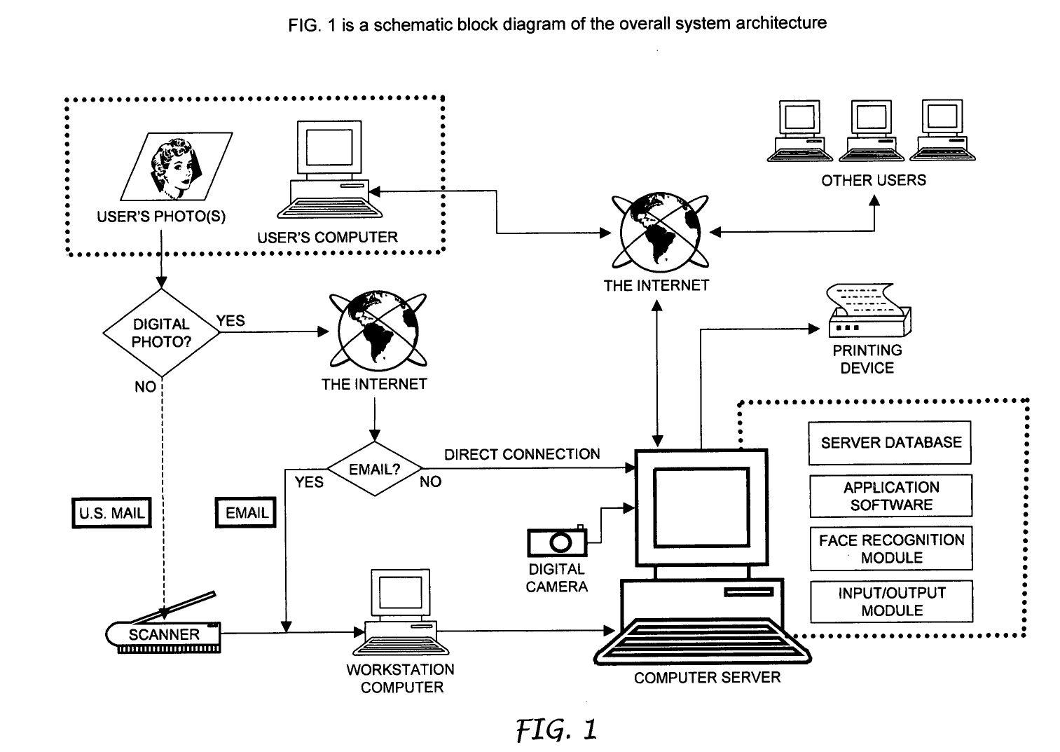

The method or arrangement for configuring and connecting the following components will be well known to those with ordinary skill in the art of computer science. FIG. 1 shows a preferred embodiment of the Biometric Compatibility Matching System 100, wherein a Computer Server 102 is connected to a network such as The Internet 116. An attached Server Database 104 is preferably some storage device such as a hard disk drive or the like with a capacity, preferably of at least 80 gigabytes. The Server Database 104 is preferably Microsoft SQL Server (trademark), Microsoft Access (trademark), or some other ODBC compliant database. The Server Database 104 contains each user's unique faceprint, along with all other data associated with the user. The Computer Server 102 contains the Face Recognition Module 108 and Application Software 106 for generating and processing unique faceprints, and applications processing. Each faceprint is capable of being created, read and processed by the Face Recognition Module 108. The Input/Output Module 110 handles all data coming into and out of the Computer Server 102.

In a preferred embodiment, the Computer Server 102 runs the Windows 2000 (trademark) operating system (or higher) on a Pentium-based (or higher) computer with a minimum 80-gigabyte hard disk and one gigabyte of random access memory (RAM). Facial Recognition software components are utilized as described below.

At another location, possibly geographically remote from that of the Computer Server 102, a Workstation Computer 130 connects to the Computer Server 102 via a secure network. The secure network connection may be via The Internet 116 or some other secure connection. A Scanner 124 or other photo-scanning device is directly connected to the Workstation Computer 130. The Workstation Computer 130 also contains the Face Recognition Module 108, Application Software 106 and Input/Output Module 110 for overall application processing. Although the Workstation Computer 130 is not essential, it's use is recommended to allow for remote processing, and to lighten the workload of the Computer Server 102.

In a preferred embodiment, the Workstation Computer 130 runs the Windows 2000 (trademark) operating system (or higher) on a Pentium-based (or higher) computer with a minimum 80-gigabyte hard disk and 512 megabytes of random access memory (RAM). The Face Recognition Module 108 used for creating and processing each faceprint would be a commercial off-the-shelf (COTS) product that utilizes a Software Development Kit (SDK) for relatively easy integration into this application. It's processing speed will also be adequate to rapidly search through a large database of image faceprints, and will possess significant fidelity for ensuring an extremely high level of precision when matching. The software can also quickly connect to and search nearly all types of databases. There are several facial recognition products currently available which meet all the above criteria, including ID-2000 (trademark) by Imagis Technologies Inc. The invention does not necessarily require any specific product, and may substitute products anytime as the technology evolves.

A user can access the Biometric Compatibility Matching System 100 either via The Internet 116 by using a User's Computer 114, or by any other form of accessing The Internet 116. There are no special requirements for the User's Computer 114, other than that it have some means of accessing The Internet 116. Other Users 128 can also access the Biometric Compatibility Matching System 100 via The Internet 116, and also only require a means of accessing The Internet 116. An alternative embodiment of the invention allows for a self-contained, standalone kiosk to function without The Internet 116, and is further described later in this section.

User's Photo(s) 118 are required and may be in either hardcopy or digital format. A digitally formatted photo may be sent via The Internet 116 either by Email 120, or uploading by Direct Connection 126. When sent by Email 120, photos are processed manually. When sent by Direct Connection 126, photos are uploaded directly to the Computer Server 102, where the Input/Output Module 110 receives them. The Application Software 106 and Face Recognition Module 108 then process them automatically. Hardcopy photos may be sent by U.S. Mail 122, or by similar means.

In operation, the Data Input process and the Search process for the Dating Matchup option of the invention are each described immediately below. The Family/Friends Matchup, Celebrity Matchup, and Pet Matchup options are described at the end of this section as alternative embodiments.

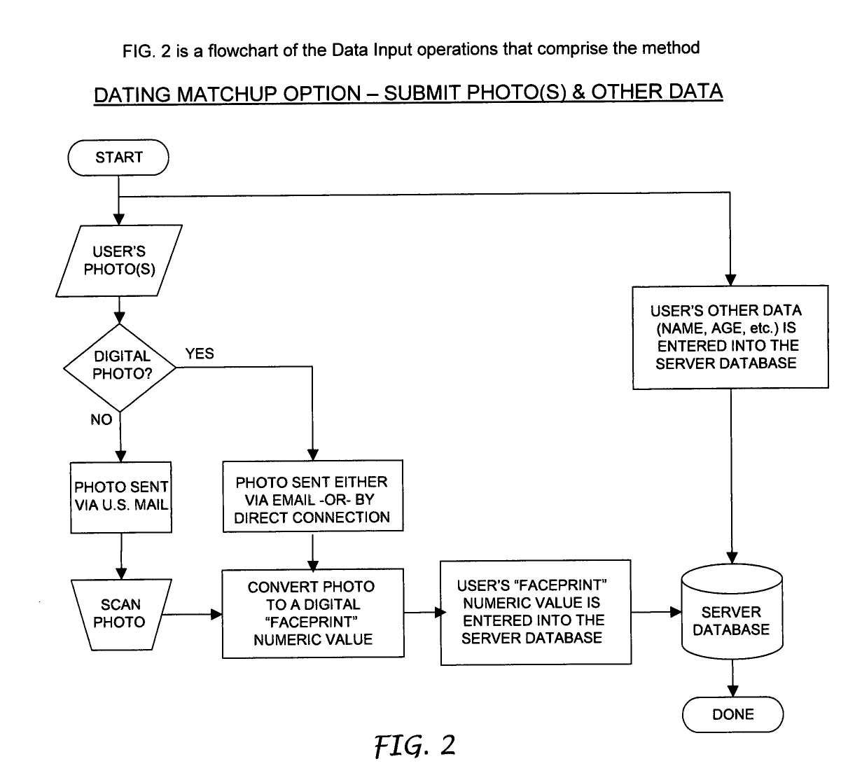

The Data Input process for the invention is described here with reference to FIG. 1 (FIG. 2 is a flowchart view of this same Data Input process).

There are several methods for sending User's Photo(s) 118. Digitally formatted photo(s) can be sent by Email 120, uploading by Direct Connection 126 via The Internet 116, or by similar means. Hardcopy photo(s) may be sent by U.S. Mail 122, or by similar means. More than one (1) photo per user may be used in order for the Face Recognition Module 108 to better determine facial characteristics. These multiple photos then create a single faceprint that more accurately represents the user's face.

The preferred method for processing User's Photo(s) 118 occurs when digital photos are sent from User's Computer 114 by Direct Connection 126 via The Internet 116. These photos are uploaded directly to the Computer Server 102 where the Input/Output Module 110 receives them. The Application Software 106 and Face Recognition Module 108 then process them automatically. The user's faceprint is then stored in the Server Database 104 immediately, with no manual processing required.

When a hardcopy photo is received for processing, the Scanner 124 or other photo-scanning device is used to convert the photo into a digital format. The now digitally formatted photo is stored on the Workstation Computer 130. The Face Recognition Module 108 located on the Workstation Computer 130 is then used to process the user's digital photo into a unique faceprint numeric value. For an already existing database of unprocessed digital photos, each photo can be sent from the Computer Server 102 to the Workstation Computer 130 through a secure network for processing. After a faceprint value has been created, it is sent via a secure network to the Computer Server 102 for storage in the Server Database 104. The user's faceprint value is stored in the appropriate field and record in the Server Database 104 for that user. Other Users 128 photos are all processed similarly. Although the Workstation Computer 130 is not essential, it's use is recommended to allow for remote processing, and to lighten the processing load of the Computer Server 102.

With reference to FIG. 2, the user's other data (name, age, etc.) is entered through the User's Computer 114 using a standard HTML form page. This is accomplished using any typical browser application such as Microsoft Internet Explorer (trademark) or Netscape (trademark). The data is transmitted via The Internet 116 to the Application Software 106 on the Computer Server 102, where it is then stored in the Server Database 104.

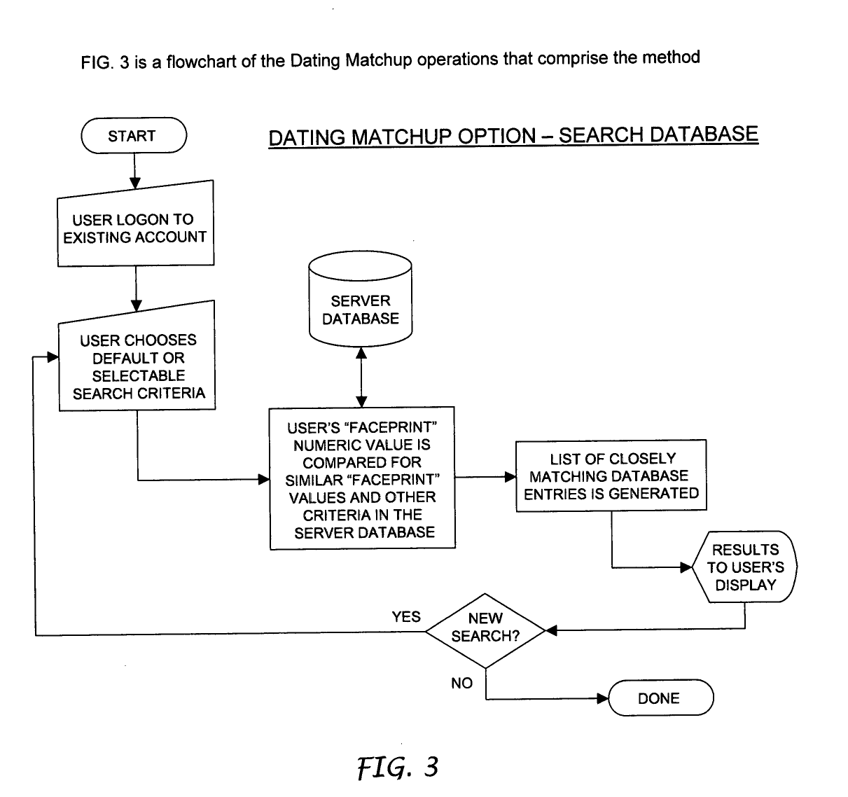

The Search process for the invention is described here with reference to FIG. 1 (FIG. 3 is a flowchart view of this same Search process). This is considered a one-to-many search since we are comparing one person (the user) to many people (all other subscribed users) in the Server Database 104.

The Server Database 104 contains information about each user who has subscribed to the Biometric Compatibility Matching System 100, as described above. In addition to a user's faceprint, the database contains other user information that can be located based on various search criteria such as gender, age, race, geographic proximity to user's home, and the like. The actual database is organized based on the underlying database query program and the hardware and software running on the system.

A user wishing to search the database must first logon and then provide search criteria. In the case of access via The Internet 116, the user's search criteria are entered using any typical browser application such as Microsoft Internet Explorer (trademark) or Netscape (trademark). These criteria can include various combinations of information stored in the Server Database 104. The search criteria for the Biometric Compatibility Matching feature would use predefined defaults, such as using a weighted system to evaluate all facial features when doing a search. As facial recognition technology evolves, user selectable search criteria such as emphasis on eyes, nose, etc., or combinations thereof, may also be used.

Then the Computer Server 102 performs a Server Database 104 search for the closest matching entries based on all specified search criteria (gender, etc.). As is the primary purpose of this invention, this includes a search for all people whose facial features most closely match the user's own facial features. Searches may be accomplished without regard to race, gender, and hair/beard styles. A user-selectable number (e.g. 50) of the closest matching entries are generated as a result of the database search. The generated matches are sorted in descending order with the closest matching results listed first. The results are then presented to the user on the User's Computer 114 display.

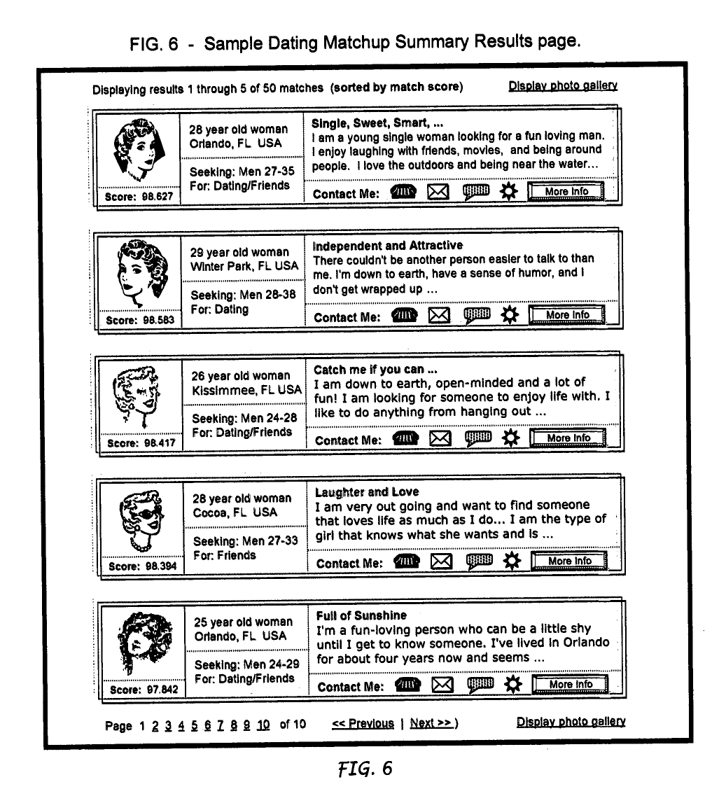

FIG. 6 is an illustration of an example Dating Matchup Summary Results page 150. This gives the user a summary of the search results. The Application Software 106 and Face Recognition Module 108 generate this page. The Input/Output Module 110 then sends these results via The Internet 116 to the User's Computer 114 display.

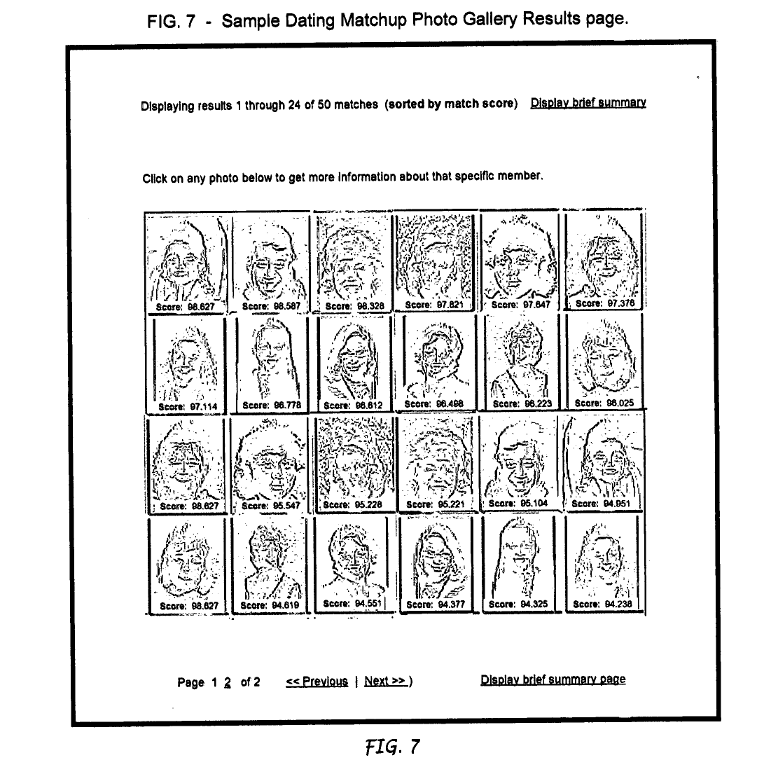

FIG. 7 is an illustration of an example Dating Matchup Photo Gallery Results page 160. This gives the user a photo gallery of the search results. The user can then click on any photo displayed to get more information about that specific member. The Application Software 106 and Face Recognition Module 108 generate this page. The Input/Output Module 110 then sends these results via The Internet 116 to the User's Computer 114 display.

The system reports the match score as a percentage for each member returned as a search result. This indicates how closely the facial features of the user and each member match. The match percentage will represent the overall match score, or as face recognition technology evolves, be on a per facial feature basis. A brief written narrative may also be generated to describe the resemblances. A user may also have the option to do a one-to-one comparison between the user and one other member. The user would do this by entering the other user's ID as the search criteria. The result for the one-to-one comparison may also be the percentage match score and/or written narrative as described above. Once a search has been completed and the results have been displayed to the user, new search criteria can be selected and a new search may be performed, or the process is done.

Alternative embodiments of the invention allow the system to be implemented as either networked (e.g. via The Internet 116) or as a non-networked, standalone kiosk. These embodiments are functionally described as follows:

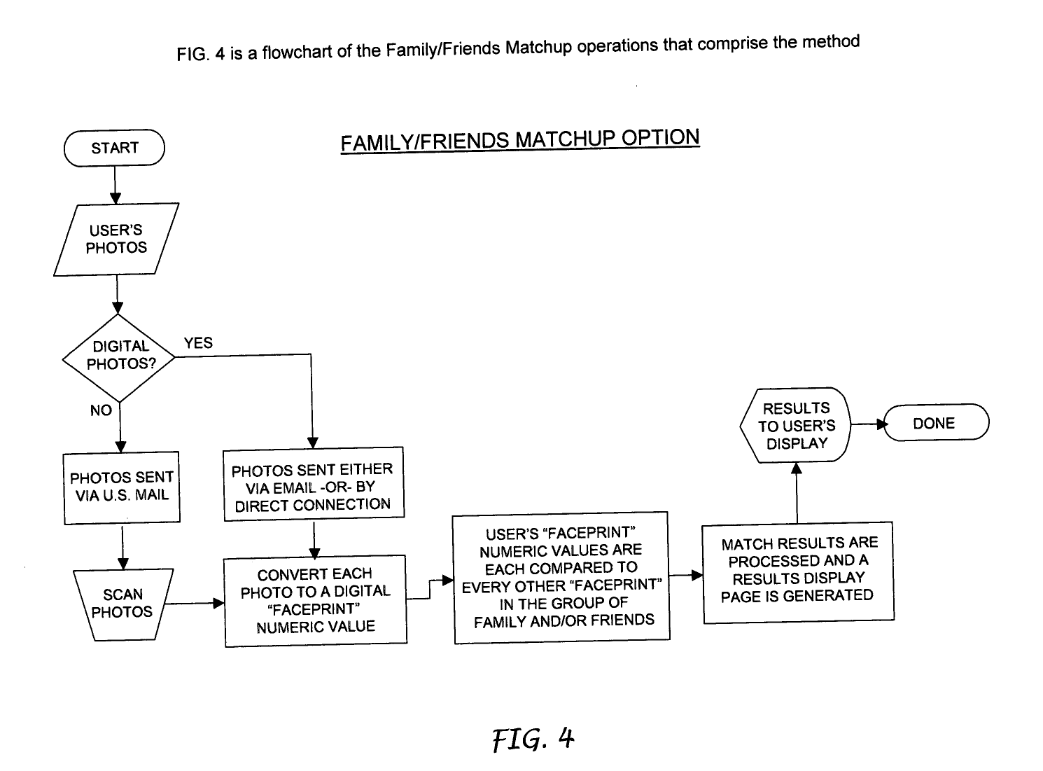

The Family/Friends Matchup process for the invention is described here with reference to FIG. 1 and FIG. 4. Since all hardware, software, and processing components are the same as previously described in the Dating Matchup section above, only the specific functionality differences will be described here.

This process allows the submission of two (2) or more photos representing a group of family and/or friends. Each of the photos is processed into a unique faceprint, and then each faceprint is compared to every other faceprint within the group to obtain a match score for each comparison. For example, a family consisting of a father, mother, son, and two daughters would have a total of 10 comparisons done in order to evaluate all possible combinations. The result would then objectively and quantitatively show how closely each group member resembles every other member within that group. In this example, 2 of the 10 comparisons would show whether the son looks more like the father or the mother.

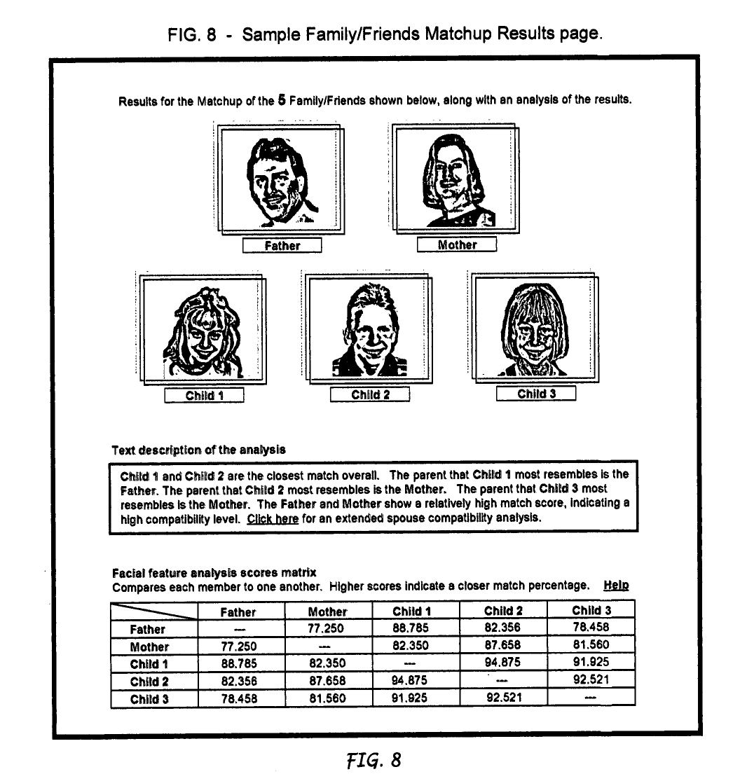

FIG. 8 is an illustration of an example Family/Friends Matchup Results page 170. This gives the user a text description of the analysis, and also a matrix of all match scores generated. The Application Software 106 and Face Recognition Module 108 generate this page. The Input/Output Module 110 then sends these results via The Internet 116 to the User's Computer 114 display.

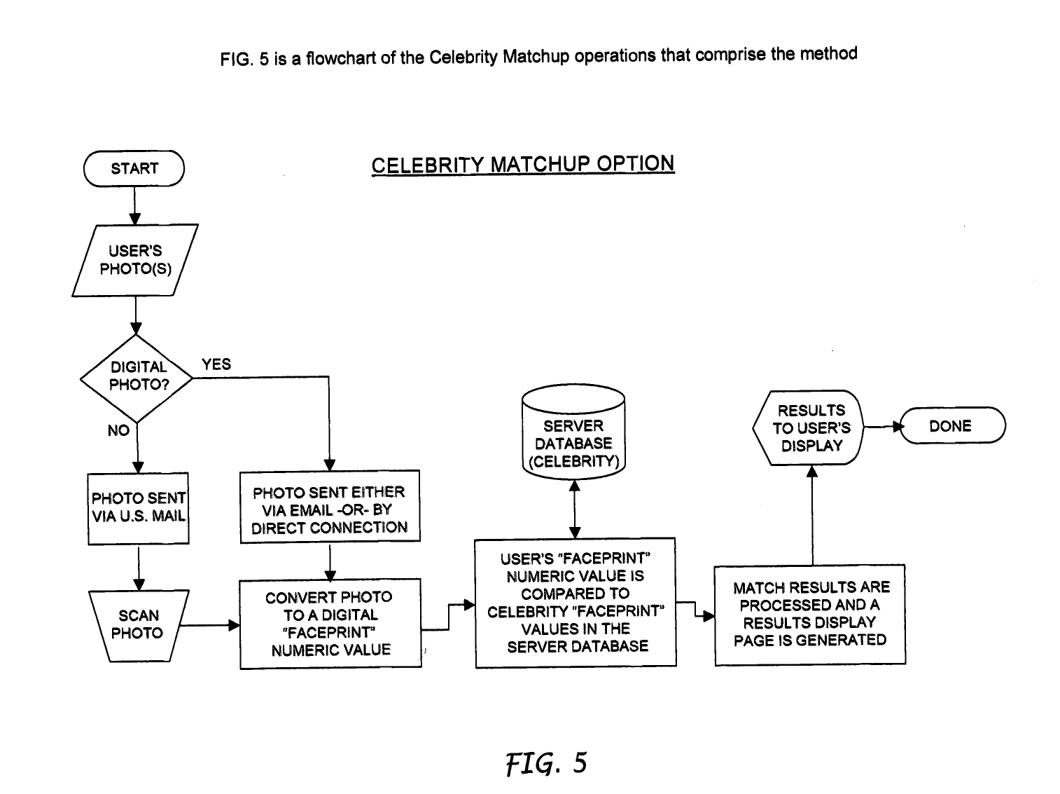

The Celebrity Matchup process for the invention is described here with reference to FIG. 1 and FIG. 5. Since all hardware, software, and processing components are the same as previously described in the Dating Matchup section above, only the specific functionality differences will be described here.

This process uses a Server Database 104 containing the photos and corresponding faceprints of well-known public figures such as athletes, politicians, actors, music and any other famous people; living or not. The database of celebrity photos may also include several photos of the same celebrity, such as a “young Paul Newman” and an “older Paul Newman”. The purpose is to compare the faceprint of the user (which may be anyone) to all or a subset of celebrity faceprints in order to determine which celebrities most closely resemble the user. A subset of all celebrities could be used to narrow the results to a specific category, such as actors. The result is a list of photos, match scores, and brief biographical data for the closest celebrity matches for the user's selected category.

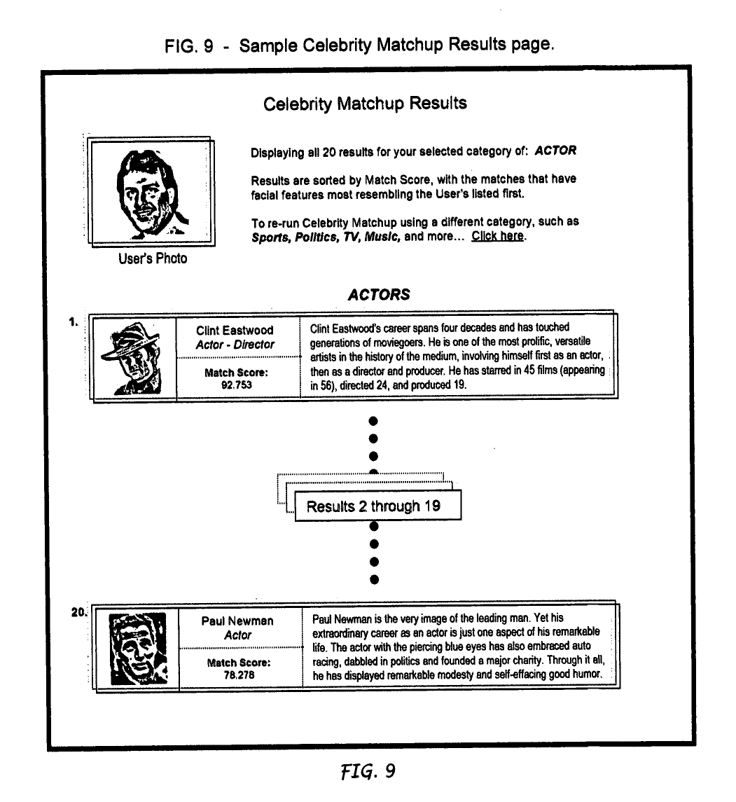

FIG. 9 is an illustration of an example Celebrity Matchup Results page 180. This gives the user a list of photos, match scores, and brief biographical data for the closest celebrity matches for the user's selected category. The Application Software 106 and Face Recognition Module 108 generate this page. The Input/Output Module 110 then sends these results via The Internet 116 to the User's Computer 114 display.

The Pet Matchup process for the invention is described here with reference to FIG. 1. Since all hardware, software, and processing components are the same as previously described in the Dating Matchup section above, only the specific functionality differences will be described here.

Ever notice how people sometimes resemble their pets? This alternative embodiment, although admittedly for amusement purposes, compares a user to a species (e.g. dog) to determine a breed (e.g. bulldog) with the closest resemblance. This allows a user to submit User's Photo(s) 118 and compare to a Server Database 104 containing the photos and corresponding faceprints of all breeds of dog. To do this, statistical data relating to a large sample of human faces is gathered to determine upper/lower boundaries for facial features such as distance between eyes, nose size, face roundness, etc. The same gathering of statistical data for all dog breeds for example, is also compiled. Then, when doing the comparison between a human and a pet species, the faceprint and statistical data would be compared to determine the closest matching breeds to the user. For example, if the user has a statistically small nose, a breed of dog with a statistically small nose would be returned as one of the match results. The result is a list of photos, match scores, and brief description for each of the closest breed matches for the user's selected species.

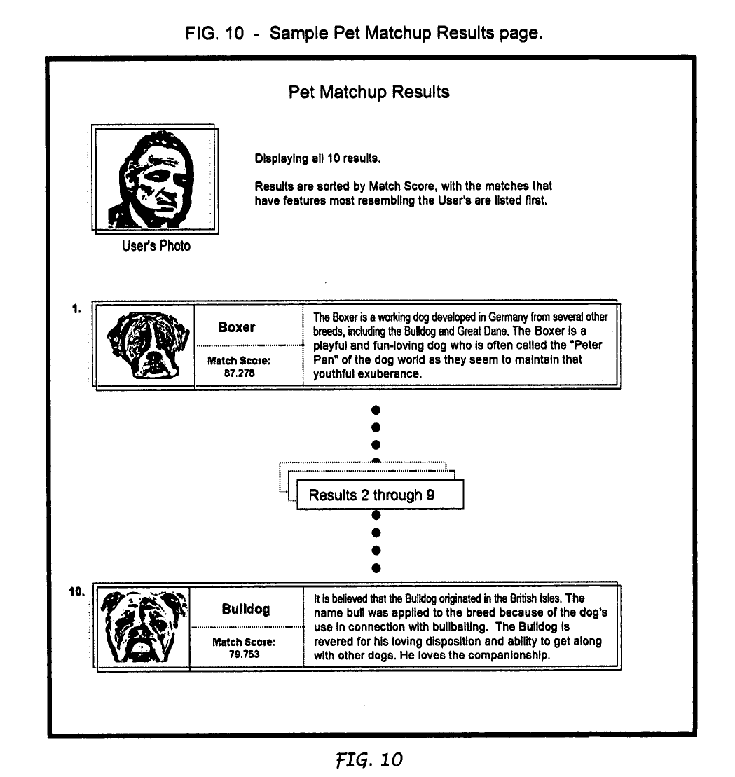

FIG. 10 is an illustration of an example Pet Matchup Results page 190. This gives the user a list of photos, match scores, and brief description of each returned result. The Application Software 106 and Face Recognition Module 108 generate this page. The Input/Output Module 110 then sends these results via The Internet 116 to the User's Computer 114 display.

Although this invention is primarily intended to be internet-based, all the functionality described herein can also be implemented as a non-networked, standalone kiosk. To accomplish this, an alternative embodiment of the physical hardware is described as follows:

Only the Computer Server 102 of FIG. 1 is required, along with any type of Digital Camera 112 capable of producing a digitally formatted photo, and a Printing Device 132. Users would have their photo taken and stored on the Computer Server 102. The Face Recognition Module 108 located on the Computer Server 102 is used to process User's Photo(s) 118 into unique faceprint numeric values. The Face Recognition Module 108 then analyzes and compares the user's facial features, and the Application Software 106 generates the results. The type of results varies depending on which functional embodiment is deployed. The system reports match percentages, indicating how closely the user's facial features match. The matching percentage can represent the overall match, or may be on a per facial feature basis. A brief written narrative may also be generated to describe the results. These results are displayed by the Input/Output Module 110 on the Computer Server 102 monitor, or printed out by a Printing Device 132. Since this embodiment is a standalone kiosk and requires no networking, its portability allows for use in locations such as shopping malls, arcades, amusement parks, and other public locations.

Although described with reference to a particular computer hardware/software implementation, the present invention can be implemented in a variety of ways. The embodiments illustrated and discussed herein are intended only to teach those skilled in the art the best way currently known by the inventor to make and use the invention. Nothing in the specification should be considered as limiting the scope of the present invention. Changes could be made by those skilled in the art to produce similar devices without departing from the spirit of my invention. As facial recognition technology evolves and progresses, the fidelity and functionality of this invention will also progress.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

1. A biometric compatibility matching system for determining compatibility and resemblance between two or more individuals by using biometric face recognition software to compare facial features comprising:
means for acquiring and storing facial images and user data, processing, and returning the results to the user;
means for storing and retrieving user data to include facial image faceprint values, name, age, gender, address, and other data, operationally connected to said means for acquiring and storing facial images and user data, processing, and returning the results to the user;
means for executing program instructions to perform various computer functions and operations, which may vary depending on the particular embodiment, functionally connected to said means for storing and retrieving user data to include facial image faceprint values, name, age, gender, address, and other data, and operationally connected to said means for acquiring and storing facial images and user data, processing, and returning the results to the user;
means for detecting facial features within images, generating and storing faceprint values, comparing a plurality of faceprint values to obtain facial match scores, and producing a list of one or more of the closest matches, functionally connected to said means for executing program instructions to perform various computer functions and operations, which may vary depending on the particular embodiment, and functionally connected to said means for storing and retrieving user data to include facial image faceprint values, name, age, gender, address, and other data;
means for receiving user's photo(s) and user's other data along with user search requests, and also for outputting the match results back to the user, functionally connected to said means for executing program instructions to perform various computer functions and operations, which may vary depending on the particular embodiment, and operationally connected to said means for acquiring and storing facial images and user data, processing, and returning the results to the user; and
means for processing a user's image(s) into a faceprint value, operationally connected to said means for receiving user's photo(s) and user's other data along with user search requests, and also for outputting the match results back to the user.

2. The biometric compatibility matching system in accordance with claim 1, wherein said means for acquiring and storing facial images and user data, processing, and returning the results to the user comprises a computer server.

3. The biometric compatibility matching system in accordance with claim 1, wherein said means for storing and retrieving user data to include facial image faceprint values, name, age, gender, address, and other data comprises a server database.

4. The biometric compatibility matching system in accordance with claim 1, wherein said means for executing program instructions to perform various computer functions and operations, which may vary depending on the particular embodiment comprises an application software.

5. The biometric compatibility matching system in accordance with claim 1, wherein said means for detecting facial features within images, generating and storing faceprint values, comparing a plurality of faceprint values to obtain facial match scores, and producing a list of one or more of the closest matches comprises a face recognition module.

6. The biometric compatibility matching system in accordance with claim 1, wherein said means for receiving user's photo(s) and user's other data along with user search requests, and also for outputting the match results back to the user comprises an input/output module.

7. The biometric compatibility matching system in accordance with claim 1, wherein said means for processing a user's image(s) into a faceprint value comprises a digital, sufficient resolution, sufficient clarity user's photo(s).

8. A method of determining compatibility and resemblance between individuals, the steps comprising:
a) storing faceprint values of a plurality of individuals;
b) providing a photograph of a user;
c) generating a faceprint value of said user;
d) comparing said faceprint value of said user with said stored faceprint values of said plurality of individuals; and
e) identifying the closest match between said photograph of said user and said faceprint values of said plurality of individuals.

9. The method of determining compatibility and resemblance between individuals in accordance with claim 8, the steps further comprising:
f) storing information representative of said plurality of individuals selected from the group: name, age, gender, address, height, hair color, eye color, ethnic background, skin coloring, body measurements, posture and education;
g) providing user information selected from the group: name, age, gender, address, height, hair color, eye color, ethnic background, skin coloring, body measurements, posture and education;
h) comparing said information of said user with said stored information of said plurality of individuals; and
i) identifying the closest match between said information of said user and said information of said plurality of individuals.

10. The method of determining compatibility and resemblance between individuals in accordance with claim 8, wherein at least one of said plurality of individuals is a celebrity.

11. The method of determining compatibility and resemblance between individuals in accordance with claim 8, wherein at least one of said plurality of individuals is a member of a pet species.

12. A method of determining compatibility and resemblance between individuals, the steps comprising:
a) storing data representative of faceprint values of a plurality of individuals;
b) providing a photograph of a user;
c) generating data representative of a faceprint value of said user;
d) comparing said data representative of a faceprint value of said user with said stored data representative of faceprint values of said plurality of individuals; and
e) identifying the closest match between said data representative of said user and said data representative of said faceprint values of said plurality of individuals.

13. The method of determining compatibility and resemblance between individuals in accordance with claim 12, wherein at least one of said plurality of individuals is a celebrity.

14. The method of determining compatibility and resemblance between individuals in accordance with claim 12, wherein at least one of said plurality of individuals is a member of a pet species.
